(19) United States
(12) Patent Application Publication
(10) Pub. No.: US 2023/0399634 A1
(43) Pub. Date: Dec. 14, 2023

(54) METHOD AND KIT FOR DNA ISOLATION

(71) Applicant: GLOBAL LIFE SCIENCES SOLUTIONS GERMANY GMBH, Dassel (DE)

(72) Inventors: Richard Martin West, Cardiff South Glamorgan (GB); Angela Sian Davies, Cardiff South Glamorgan (GB); Malcolm John Hatcher, Cardiff South Glamorgan (GB); Monika Seidel, Cardiff South Glamorgan (GB)

(21) Appl. No.: 17/793,635

(22) PCT Filed: Jan. 19, 2021
(86) PCT No.: PCT/EP2021/051046

§ 371 Date: Jul. 18, 2022

(30) Foreign Application Priority Data
Jan. 24, 2020 (GB) 2001034.4

(51) Int. Cl. C12N 15/10; C12Q 1/6806

(52) U.S. Cl. CPC C12N 15/1013; C12Q 1/6806

(57) ABSTRACT

A method for isolating cell-free DNA from liquid body sample, comprising the following steps: a) Providing liquid body sample; b) Adding to said sample: a solid phase capable of binding DNA; a binding buffer comprising a detergent and a chaotropic agent; and 2-propanol, to form a binding mixture thereof; c) Washing the solid phase to remove unbound material; and d) Eluting bound cell-free DNA, wherein the majority of the eluted DNA is <400 bp.

### TECHNICAL FIELD

The present invention relates to an improved method and system for isolating cell-free DNA (cfDNA) present in a liquid body sample. More specifically, the present invention relates to a method and system for isolating cfDNA from blood plasma with size selection, to facilitate enrichment and recovery of small fragment cfDNA while at the same time minimizing recovery of high molecular weight, larger genomic DNA (gDNA) fragments.

### BACKGROUND

Fragmented cfDNA molecules were first discovered in the human circulatory system in 1948 by Mandel and Metais. cfDNA also known as circulating free DNA or circulating cell-free DNA, are DNA fragments released into the bloodstream by the cells. Several mechanisms of the release of cfDNA molecules in blood have been proposed including necrosis, apoptosis, phagocytosis, active cellular secretion, exosome release, pyroptosis, mitotic catastrophe and autophagy, resulting in the presence of a cfDNA population with diverse physical properties in circulation. In healthy individuals, cfDNA fragments vary between 100-250 bp with the most prevailing size of 166 bp that corresponds a nucleosome complex of DNA molecule bound to the histone core. cfDNA can be used to describe various forms of fragmented DNA circulating freely in the bloodstream such as cell-free fetal DNA (cffDNA), circulating tumor DNA (ctDNA) or circulating cell-free mitochondrial DNA (ccf mtDNA).

Clinical significance of cfDNA was recognized when researchers observed differences between the characteristics of cfDNA from healthy and diseased individuals. Numerous studies have demonstrated that cancer patients generally have high levels of cfDNA in comparison to healthy subjects. Elevated levels of cfDNA in cancer patients is thought to be caused by excessive DNA release by apoptotic and necrotic cells and/or cfDNA accumulation due to chronic inflammation and excessive cell death. In healthy individuals, cfDNA levels are predominantly low, however they can get temporarily elevated after strenuous exercise. It has also been demonstrated that the size of cfDNA fragments that originate from tumor cells are shorter than cfDNA fragments that originate from non-malignant cells. Similarly, cfDNA of fetal origin contains a higher proportion of DNA smaller than 150 bp. Increased proportion of smaller fragments has also been reported in autoimmune disease and in donor derived fraction post transplantation. Thus, size-selection of smaller cfDNA fragments could be used to increase the amount of target cfDNA fragments (i.e. tumor derived cfDNA in cancer diagnostics or fetal cfDNA in noninvasive prenatal testing). cfDNA in cancer patients bear the unique genetic and epigenetic alterations that are characteristic of the tumor from which they originate. Genetic analysis and molecular profiling of cfDNA thus presents a promising clinical potential for cancer detection, prognosis, staging, monitoring and therapy selection. cfDNA as a biomarker for cancer management has been successfully demonstrated by two FDA-approved applications for cfDNA assays in routine clinical practice, namely the cobas EGFR Mutation Test v2 for lung cancer patients and Epi proColon, a colorectal cancer screening test based on the methylation status of the SEPT9 promotor. Fetal derived cfDNA present in maternal blood has also been successfully used to detect fetal abnormalities. cfDNA analysis has also shown potential for clinical use in organ transplant, autoimmune diseases and sepsis where cfDNA fraction is enriched in smaller DNA molecules.

In the blood of cancer patients, cfDNA originates from multiple sources including not just cancer cells but also cells from the tumor micro-environment and other non-cancer cells from various parts of the body. DNA from cancer cells is released most prominently by the mechanisms of apoptosis, necrosis, and active secretion. Apoptosis causes the systematic cleavage of chromosomal DNA into multiples of 160-180 bp stretches, resulting in the extracellular presence of mono-nucleosomes and poly-nucleosomes. The majority of cfDNA produced by apoptosis has a size of 167 bp (147 bp of DNA wrapped around a nucleosome plus a linker DNA of around 20 bp that links two nucleosome cores).

Solid tumor biopsies are expensive and invasive, making them less than ideal for patients who are older or very young. On the other hand, cfDNA analysis as a disease biomarker can be done using non-invasive liquid biopsy which utilizes a liquid body sample from the patient like blood plasma, urine or serum. The amount of ctDNA in the whole pool of cfDNA may vary widely among the patients, cancer type, and cancer stage, from 0.01% to 90% in advanced metastasis. There is a prevailing consensus that ctDNA is fragmented to a higher extent than cfDNA derived from healthy cells and has shorter fragment size (less than 150 bp). Both low abundance and shorter fragment size of ctDNA presents a serious challenge to the isolation and further analysis of cfDNA. In addition, intra-tumoral genetic heterogeneity is yet another challenge in clinical oncology where identification of minor sub-clonal populations is essential for detection of emerging chemoresistance, minimal residual disease, and non-invasive monitoring of disease progression. The detection limit becomes negatively affected by the presence of contaminating high molecular weight gDNA that may be present in the plasma, originating from lysed blood cells. Therefore, it is important to select a cfDNA extraction method that not only delivers a high yield of cfDNA but also allows for efficient recovery of shorter cfDNA fragments and negatively selects against high molecular weight DNA. To detect some rare low-level resistance mutation, one is more likely to detect it when tumor originating cfDNA is enriched in the sample and blood cell-derived gDNA background is reduced to minimum. cfDNA is thus usually purified from the plasma or serum which is devoid of white blood cells (WBCs) to prevent gDNA contamination resulting from WBC lysis. gDNA contamination would dilute out the tumor cfDNA, preventing detection of rare variants. As increased fragmentation of cfDNA has been widely reported for fetal-derived cfDNA, donor-derived cfDNA following organ transplantation and in autoimmune disease, size selection-based cfDNA extraction provides a clear advantage outside cancer diagnostics.

### SUMMARY OF THE INVENTION

The object of the present invention is to provide an improved and size-selective method for isolation of cfDNA from liquid body sample like blood plasma.

The distinctive advantage of the method is that it allows for efficient isolation of main cfDNA fraction together with smaller, highly degraded fragments over any high molecular weight gDNA that would be perceived as a contaminant. This size dependent DNA binding allows for the specific enrichment of extracted cfDNA in the fraction of interest, e.g. tumor derived cfDNA in cancer or fetal cfDNA during prenatal testing in pregnancies with suspected aneuploidy. This makes the method of the invention highly suitable for liquid biopsy-based diagnostics.

Another advantage of the method is that it requires a very small quantity of input plasma sample ranging from 0.5 ml-4 ml.

Another advantage of the method is that it is quick and cfDNA isolation procedure can be completed in less than 2 hours to yield high quality cfDNA suitable for downstream applications.

According to an aspect of the invention, a method for isolating cell-free DNA from liquid body sample comprises the following steps:
a) Providing liquid body sample;
b) Adding to said sample:
   a solid phase capable of binding DNA;
   a binding buffer comprising a detergent and a chaotropic agent; and
   2-propanol,
to form a binding mixture thereof;
c) Washing the solid phase to remove unbound material; and
d) Eluting bound cell-free DNA,
wherein the majority of the eluted DNA is <400 bp.

According to another aspect of the invention, a method for size-selective isolation of cell-free DNA from liquid body sample, comprises the following steps:
a) Providing liquid body sample;
b) Adding to said sample:
   an aqueous suspension of silica coated magnetic microbeads capable of binding DNA;
   a binding buffer comprising guanidinium thiocyanate and non-ionic surfactant such as Triton X-100; and
   2-propanol,
to form a binding mixture thereof such that said binding mixture comprises a non-ionic surfactant such as Triton X-100 at around 20-30% w/v, guanidinium thiocyanate at around 1.5-2.5 M and 2-propanol at around 15-25% v/v;
c) Incubating said binding mixture at room temperature for about 10-30 minutes to promote binding of cell-free DNA to the magnetic microbeads;
d) Washing the magnetic microbeads with one or more wash buffers comprising ethanol;
e) Adding elution buffer to the washed magnetic beads of step d) to release the cell-free DNA bound to the magnetic microbeads in solution; and
f) Optionally analysing or quantifying the cell-free DNA obtained in step e).

According to another aspect of the invention, guanidinium thiocyanate and Triton X-100 are used to form a binding buffer composition for size-selective binding of cell-free DNA present in blood plasma to silica-coated magnetic microbeads formulated in an aqueous suspension at 20-200 mg/ml, wherein said binding buffer is intended to be brought into contact with 2-propanol, blood plasma and magnetic microbeads to form a binding mixture comprising: around 1.5-2.5 M guanidinium thiocyanate; around 20-30% w/v of Triton X-100; around 15-25% v/v of 2-propanol; and around 25-40% v/v of blood plasma.

According to another aspect of the invention, a kit comprising silica coated microbeads capable of binding 50-400 bp DNA from a body sample in the presence of guanidinium thiocyanate, Triton X-100 and 2-propanol is described.

More advantages and benefits of the present invention will become readily apparent to the person skilled in the art in view of the detailed description below.

### BRIEF DESCRIPTION OF DRAWINGS

The invention will now be described in more detail with reference to the appended drawings, wherein:

FIG. 1 illustrates the general methodology that is used to extract cfDNA from whole blood in accordance with the method of the invention.

FIG. 2a shows a Bioanalyzer plot showing size dependent recovery of DNA fragments using the method of the invention.

FIG. 2b shows percent recovery of selected low molecular weight DNA fragments and high molecular weight DNA fragments using the method of the invention.

FIG. 3 shows a Bioanalyzer plot for Prep. Nos. 10A-10D to show the effect of varying proportion of 2-propanol in the binding mixture.

FIG. 4 shows a Bioanalyzer plot for Prep. Nos. 10E-10H to show the effect of varying proportion of 2-propanol in the binding mixture.

FIG. 5 shows a Bioanalyzer plot for Prep. Nos. 13A Repeat and 13G Repeat to show the effect of varying proportion of 2-propanol in the binding mixture.

FIG. 6 shows a Bioanalyzer plot showing effect of varying proportion of 2-propanol in the binding mixture.

FIG. 7 shows an enlarged view of a portion of FIG. 6 pertaining to low molecular weight DNA fragments.

FIG. 8 shows another enlarged view of a portion of FIG. 6 pertaining to high molecular weight DNA fragments.

FIG. 9 shows a Bioanalyzer plot showing the effect of varying proportion of Triton X-100 (8.8% and 11.1%) in the binding mixture.

FIG. 10 shows a Bioanalyzer plot showing the effect of varying proportion (8.8% and 4.5%) of Triton X-100 in the binding mixture.

FIG. 11 shows an electropherogram to show the effect of plasma components on size selection.

FIG. 12 shows a Bioanalyzer plot which shows the scalability of the cfDNA isolation method of the invention for varying plasma input volumes.

FIG. 13 shows a Bioanalyzer plot showing cfDNA recovery profile using blood plasma collected in standard EDTA tubes.

FIG. 14 shows the size selection advantage of the method of the invention in cancer mutation detection over a commercial kit with no size selection.

To more clearly and concisely describe and point out the subject matter of the claimed invention, definitions are provided hereinbelow for specific terms used throughout the present specification and claims. Any exemplification of specific terms herein should be considered as a non-limiting example.

The terms “comprising” or “comprises” have their conventional meaning throughout this application and imply that the agent or composition must have the essential features or components listed, but that others may be present in addition. The term ‘comprising’ includes as a preferred subset “consisting essentially of” which means that the composition has the components listed without other features or components being present.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### List of Abbreviations

cfDNA: cell-free DNA
cffDNA: cell-free fetal DNA
ccf mtDNA: circulating cell-free mitochondrial DNA
PBS: phosphate-buffered saline
GuSCN: guanidinium thiocyanate
bp: base pair
EDTA: Ethylenediaminetetraacetic acid

### NGS: Next Generation Sequencing

### SDS: Sodium Dodecyl Sulfate

gDNA: Genomic DNA

### WBC: White Blood Cell

### PCR: Polymerase Chain Reaction

ddPCR: Digital Droplet PCR

### List of Equipment Used in Examples

1. Centrifuges that accommodate 15 mL centrifuge tubes and 1.5 mL micro-centrifuge tube
2. Standard laboratory shakers/mixers, for example the Eppendorf™ Thermomixer to accommodate 15 mL centrifuge tubes and 1.5 mL microcentrifuge tubes.
3. Incubator
4. Vortex mixer
5. 15 mL centrifuge tubes and 1.5 mL microcentrifuge tubes
6. cfDNA stabilizing tubes for blood collection
7. Pipette tips with aerosol barrier
8. Magnetic racks, to fit 15 mL centrifuge tubes and 1.5 mL microcentrifuge tubes, e.g. MagRack 6 and MagRack Maxi (GE Healthcare).
9. Bioanalyzer 2100 (Agilent)

All tubes and pipette tips used were of DNase-free grade. Good laboratory practices were followed to avoid sample contamination.

### DETAILED DESCRIPTION

The cfDNA isolation method of the invention allows for rapid extraction and purification of cfDNA from small quantities of liquid body sample such as blood plasma ranging from 0.5 ml-4 ml and provides high-resolution cfDNA size selection. The method has been specifically designed to select for short-fragment cfDNA (50 bp-400 bp) over longer high-molecular weight contaminating gDNA. The isolation procedure of the invention can be completed in less than 2 hours to yield high quality cfDNA suitable for downstream applications such as PCR, digital droplet PCR (ddPCR), genotyping and next generation sequencing (NGS).

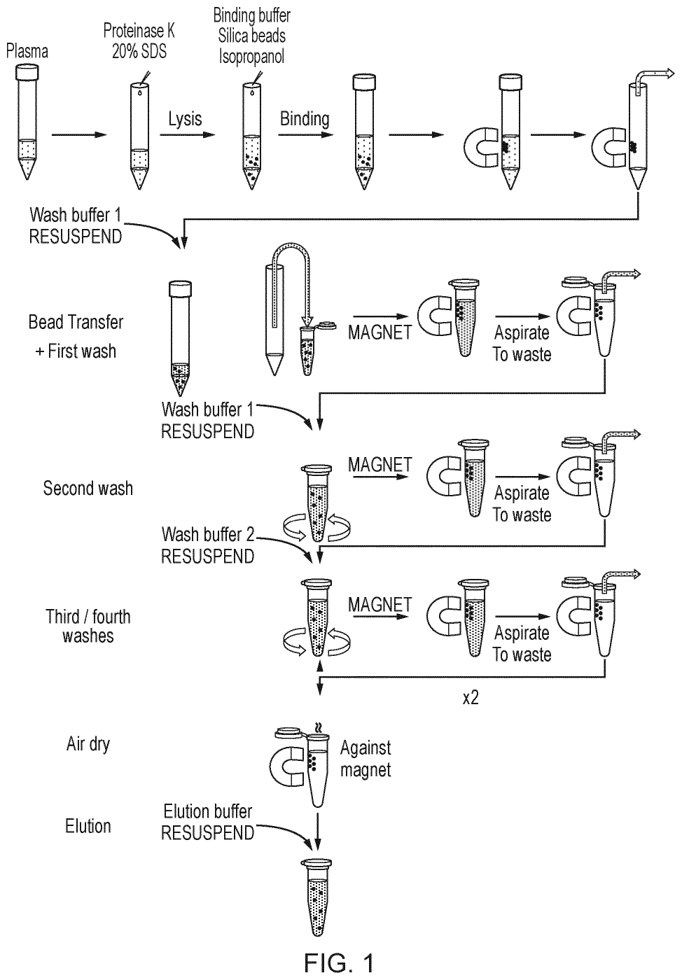

FIG. 1 illustrates the general methodology that is used to extract cfDNA from whole blood in accordance with the method of the invention. In order to ensure the highest quality and quantity of cfDNA, the blood sample is typically collected in cfDNA stabilizing tubes, for example, Streck cfDNA blood collection tubes. Streck cfDNA blood collection tube is a blood collection device with a stabilization reagent that preserves cfDNA in a blood sample for up to 14 days at room temperature by stabilizing nucleated blood cells in blood and preventing cellular DNA release into plasma. As a person skilled in the art would appreciate, EDTA tubes or Heparin tubes could be used as alternatives. After blood collection, the collection tubes are stored at ambient temperature until further processing to obtain plasma. The collection tubes are centrifuged at a lower speed of 1600×g for 10 minutes at 20° C. to separate plasma from intact blood cells. The upper plasma fraction (approx. 4-5 mL per 10 mL blood) is aspirated into a fresh tube without disturbing the buffy coat layer positioned between plasma and sedimented red blood cells layer. The tubes are then re-centrifuged at a high speed of 16000×g for 10 minutes at 20° C. to get rid of cell debris and other contaminants to obtain clear plasma. The clear plasma fraction is aspirated into a fresh tube leaving any cellular residue behind. As a skilled person would know, for larger volumes of plasma and to ensure sample homogeneity, the general recommendation is to pool the individual aspirations, then re-aliquot into convenient units for cfDNA isolation e.g., 2.0 mL units. Plasma is then processed for cfDNA isolation immediately or stored in aliquots at −20° C./−80° C. until required. Purified cfDNA may be stored at 2-8° C. for a short period if being used directly for analysis and/or downstream molecular biology applications. For longer periods of storage −20° C. or −80° C. is recommended.

The predominant type of cfDNA found in plasma is derived from the nuclear genome and has a fragment size that corresponds to a single nucleosome. These macromolecular complexes need to be dissociated in order to release cfDNA and promote binding of cfDNA to a DNA binding solid phase. In an embodiment of the invention, the solid phase was preferably silica coated magnetic microbeads where the silica bead surface is directly involved in DNA binding via surface silane (Si—OH) groups. Some of the cfDNA is also believed to be encapsulated in lipid vesicles and needs to be released prior to the binding step. The release of cfDNA from these diverse macromolecule complexes and lipid vesicles is achieved by using a combination of chaotropic agents and detergents. Chaotropic agents disrupt the nucleosomal unit to release cfDNA and detergents help to solubilize and denature proteins to release non-covalently bound cfDNA. Where the blood is collected in Streck cfDNA blood collection tubes, proteinase K treatment is additionally required to reverse the effects of Streck cfDNA stabilization chemistry by removing the crosslinks which would otherwise prevent efficient recovery of cfDNA during the isolation process. As a person skilled in the art would appreciate, Proteinase K treatment might not be required when using other blood collection tubes. Denatured contaminants are then removed by subsequent washing of the silica beads with wash buffers followed by air-drying of the silica beads. The purified cfDNA is then eluted from the silica beads using an elution buffer. In a preferred embodiment of the invention, SeraSil-Mag 700 beads by GE Healthcare Life Sciences were used for binding the released cfDNA, GuSCN was used as the chaotropic agent and 20% SDS (sodium dodecyl sulfate) was used as the detergent. As would be appreciated by the skilled person, any other DNA binding solid phase could be used instead of silica beads, for example,

the solid phase could be beads, particles, sheets and membranes having inherent DNA binding or added DNA binding capability.

As the levels of cfDNA encountered in blood plasma are very low, a significant volume reduction is needed during the isolation process to generate sufficient concentration of cfDNA for analysis. Efficient binding of the cfDNA from blood plasma, gentle washing and minimal elution are key to providing purified cfDNA that is suitable for downstream applications. Due to typically low levels of cfDNA in the final extract, UV-absorbance based analysis is not usually recommended.

Instead, cfDNA concentration is evaluated using qPCR or fluorescence-based methods such as Qubit™ (Invitrogen™). Qubit™ dsDNA HS Assay Kit, that is compatible with any fluorometer or fluorescence plate reader, allows for accurate estimation of total DNA concentrations down to 10 pg/μL. To assess the quality and yield of cfDNA in addition to the presence of gDNA, assessment can be performed using the Agilent 2100 Bioanalyzer system, with the Agilent High Sensitivity DNA Analysis Kit.

Protocol for Purification of cfDNA from 1.0-4.0 mL Blood Plasma

### Example 1

Whole blood sample collected in Streck cfDNA blood collection tubes was processed to separate the plasma as described above. The following steps of the cfDNA isolation method were then performed to obtain purified cfDNA from the plasma sample. Each step was performed using three different input plasma volumes of 1 ml, 2 ml and 4 ml of the plasma sample.

### Step 1. Lysis

This step is performed to release cfDNA from macromolecular complexes and to reverse the Streck DNA stabilization chemistry. Proteinase K (20 mg/mL) solution and plasma sample were added into a 15 mL Streck cfDNA blood collection tube and mixed by brief vortexing. 20% Sodium Dodecyl Sulfate (SDS) was then added into the tube. Either the proteinase K or the plasma may be added to the tube first. However, 20% SDS should not be allowed to contact the proteinase K solution directly to prevent enzyme inactivation. The tube was pulse vortexed 2-3 times and the contents mixed thoroughly by vortexing for 15 seconds. The tube was then incubated at about 55-65° C. for around 20-30 minutes. Table 1 below shows different plasma input volumes that were used and the corresponding quantities of proteinase K and 20% SDS.

TABLE 1

Plasma input volume

| Reagent | 1 mL | 2 mL | | 4 mL | |
|---|---|---|---|---|---|
| Proteinase K | 20 μL | 40 | μL | 80 | μL |
| 20% SDS | 50 μL | 100 | μL | 200 | μL |

Step 2: Binding Mixture Preparation

Magnetic microbeads (Sera-Sil Mag 700 by GE Healthcare Life Sciences) were fully resuspended by vortexing before dispensing. A binding mixture was prepared by combining the plasma of step 1, a binding buffer, an aqueous suspension of the magnetic beads and 2-propanol. In this example, a composite reagent was first prepared by combining the binding buffer, aqueous suspension of the magnetic beads and 2-propanol. This pre-mixed composite reagent was then added to the plasma containing tube of step 1 and mixed thoroughly by pulse vortexing to form the binding mixture. A person skilled in the art would appreciate that the binding mixture could also be prepared by adding the binding buffer, magnetic beads and 2-propanol one by one into the plasma containing tube followed by thorough pulse vortexing rather than using the pre-mixed composite reagent. In an embodiment of the invention, the microbeads and the binding buffer are added to the plasma sample before adding 2-propanol.

The relative amount of each component in the binding mixture is critical for the maximum cfDNA recovery and minimum binding of gDNA. Table 2 below shows the quantities of the pre-mixed composite reagent used corresponding to the three different input plasma volumes to form the binding mixture. Table 3 shows quantities of individual components of the composite reagent as shown in Table 2.

TABLE 2

| | Plasma input volume | | |
|---|---|---|---|
| | 1 mL | 2 mL | 4 mL |
| Composite | 2.1 mL | 4.2 mL | 8.4 mL |
| Reagent | | | |

TABLE 3

| Composite Reagent | Plasma input volume | | | |
|---|---|---|---|---|
| Component | | 1 mL | 2 mL | 4 mL |

| | | | | | | |
|---|---|---|---|---|---|---|
| Binding buffer | 1.45 | mL | 2.90 | mL | 5.80 | mL |
| Magnetic beads | 7.5 | μL | 15 | μL | 30 | μL |
| 2-Propanol | 0.70 | mL | 1.40 | mL | 2.80 | mL |

The binding buffer is typically composed of a detergent and a chaotropic agent. In this example, Triton X-100 was used as the detergent and GuSCN was used as the chaotropic agent. Triton X-100 is a non-ionic surfactant, for example, a surfactant having hydrophilic polyethylene oxide chain and an aromatic hydrocarbon lipophilic or hydrophobic group (C₁₄H₂₂O(C₂H₄O)n where n=9-10). As would be appreciated by a person skilled in the art, other detergents and chaotropic agents could be used with a similar effect. Some examples of alternate detergents are Triton X-114, Nonidet P-40 and Igepal CA-630. An example of an alternate chaotropic agent is sodium perchlorate.

The tube containing the binding mixture was then incubated in a thermomixer (25° C., 1400 rpm) for 10 minutes after which it was briefly spun and placed on a magnetic rack for at least 5 minutes. Once the beads containing bound cfDNA were collected against the magnet to form a bead pellet, the clear supernatant comprising of denatured proteins/lipids was carefully aspirated to waste.

### Step 3: Bead Transfer

The tube was removed from the magnetic rack and 400 μL of Wash Buffer 1 was added to the tube directly on the bead pellet. Wash Buffer 1 was composed of 50% of ethanol and 50% of a solution containing GuSCN at around 2.0M and a non-ionic surfactant such as Triton X-100 at about 22% w/v. The beads were fully resuspended by pulse vortexing and brief spinning. The bead suspension was pipetted up and down and the content of the tube was transferred into a 1.5 mL microtube. Due to the liquid viscosity, the content of the tip was expelled slowly to ensure complete transfer of the bead suspension. A second aliquot of Wash Buffer 1 (400 μL) was added to the tube. The tube was vortexed, briefly spun and the content transferred to the same 1.5 mL microtube. The microtube was then placed on a magnetic rack for 1 minute to allow the beads to collect against the magnet before discarding the supernatant.

### Step 4: Bead Washes

The microtube was removed from the magnetic rack and 700 μL of Wash Buffer 1 was added to the microtube. The microtube was incubated in the thermomixer at 25° C./1400 rpm for 1 minute, vortexed and then briefly spun. The microtube was then placed on the magnetic rack for 1 minute before discarding the supernatant. The microtube was removed from the magnetic rack and 700 μL of Wash Buffer 2 was added to the microtube. Wash Buffer 2 was composed of 80% of ethanol and 20% of a solution containing tris-HCl at around 10 mM, EDTA at around 1.0 mM and a polysorbate-type non-ionic surfactant such as TWEEN-20 at around 0.5% w/v. As a skilled person would appreciate, alternate non-ionic surfactants could also be used with a similar effect. Some of the examples are Tween-80 or Tween-60. Alternatively, the surfactant could be omitted altogether. The microtube was incubated in a thermomixer at 25° C./1400 rpm for 1 minute, vortexed and briefly spun. The microtube was then placed on the magnetic rack for 1 minute before discarding the supernatant. Another round of washing was done using the Wash Buffer 2.

### Step 5: Air Drying

The microtube was briefly spun to collect any residual Wash Buffer 2 at the bottom of the microtube. The microtube was placed on the magnetic rack for 1 minute to allow the beads to collect against the magnet. The clear residual supernatant was carefully removed from the very bottom of the microtube using a small pipette tip. The bead pellet was then allowed to air-dry for minutes while on the magnetic rack.

### Step 6: Elution

The microtube was removed from the magnetic rack. Elution buffer was added to the microtube and mixed well by vortexing to ensure the bead pellet was fully resuspended. The elution buffer contained tris-HCl at around 10 mM and EDTA at around 0.5 mM and the pH adjusted to 8.0. The microtube was incubated in the thermomixer at 25° C./1400 rpm for 3 minutes and briefly spun to bring the bead suspension to the bottom of the tube. The tube was placed on the magnetic rack for 1 minute to allow for the beads to collect against the magnet. Once the beads were collected against the magnet, the supernatant containing the isolated cfDNA was carefully transferred into a fresh microtube. Table 4 below shows the amount of elution buffer used corresponding to the three different volumes of input plasma.

TABLE 4

| | Plasma input volume | | |
|---|---|---|---|
| Reagent | 1 mL | 2 mL | 4 mL |
| Elution buffer | 15 μL | 30 μL | 60 μL |

Protocol for Purification of cfDNA from 500 μL (0.5 ml) Plasma

### Example 2

Whole blood sample was processed to separate the plasma as described previously. The following steps of the cfDNA isolation method were then performed to obtain purified cfDNA from 0.5 ml of plasma sample.

### Step 1: Lysis

10 μL of Proteinase K (20 mg/mL) and 0.5 ml of plasma were added into a 2 ml microcentrifuge tube and mixed by brief vortexing. 25 μL of 20% SDS was then added to the tube. Either the Proteinase K or the plasma may be added to the tube first. However, 20% SDS should not be allowed to contact the Proteinase K solution directly to prevent enzyme inactivation. The tube was pulse vortexed 2-3 times and contents mixed thoroughly by vortexing for 15 seconds. The tube was then incubated at about 55-65° C. for around 20-30 minutes.

### Step 2: Binding Mixture Preparation

The magnetic microbeads (Sera-Sil Mag 700 by GE Healthcare Life Sciences) were fully resuspended by vortexing before dispensing. A composite reagent was prepared by combining the below three components and mixing thoroughly by pulse vortexing.

1. 0.725 mL Binding buffer×(No. of samples to be processed+10%)

2. 0.35 mL 2-Propanol×(No. of samples to be processed+10%)

3. 3.75 μL Magnetic Bead suspension (No. of samples+10%)

1.05 ml of freshly prepared composite reagent was added into the plasma containing tube of step 1 and contents mixed thoroughly by pulse vortexing to prepare the binding mixture. As mentioned in Example 1 above, the binding buffer, magnetic bead suspension and 2-propanol could be added one by one to the plasma containing tube of step 1 instead of using the pre-mixed composite reagent.

The tube was then incubated in the thermomixer at 25° C./1400 rpm for 10 minutes. The tube was then briefly spun and placed on a magnetic rack for at least 5 minutes. Once the beads containing bound cfDNA were collected against the magnet, the clear supernatant was carefully aspirated to waste.

### Step 3: Bead Washes

The tube was removed from the magnetic rack and 700 μL of wash buffer was added into the tube. Multiple washing rounds were performed using Wash buffers 1 and 2 as shown in Table 5 below.

TABLE 5

| Wash round | Wash buffer |
|---|---|
| 1^{st} | Wash buffer 1 |
| 2^{nd} | Wash buffer 1 |
| 3^{rd} | Wash buffer 2 |
| 4^{th} | Wash buffer 2 |

Wash Buffers 1 and 2 used were as described in Example 1 above. The tube was incubated in a thermomixer at 25° C./1400 rpm for 1 minute followed by vortexing and brief spinning. The tube was then placed on the magnetic rack for 1 minute before discarding the supernatant.

### Step 4: Air Drying

The tube was briefly spun to bring any residual Wash buffer 2 droplets to the bottom of the tube. The tube was then placed on a magnetic rack for 1 minute to allow for the beads to collect against the magnet. Clear residual supernatant was carefully removed from the very bottom of the microtube using a small pipette tip and the bead pellet was allowed to air-dry for 5 minutes while on the magnetic rack.

### Step 5: Elution

The tube was removed from the magnetic rack. 15 μL of elution buffer was added into the tube and the contents of the tube mixed well by vortexing to ensure the bead pellet was fully resuspended. The elution buffer used was the same as described in Example 1 above. The tube was incubated in the thermomixer at 25° C./1400 rpm for 3 minutes and then briefly spun to bring bead suspension to the bottom of the tube. The tube was placed on the magnetic rack for 1 minute to allow for the beads to collect against the magnet. Once the beads were collected against the magnet, the supernatant containing the isolated cfDNA was carefully transferred into a fresh microcentrifuge tube.

### Recovery Versus Fragment Size

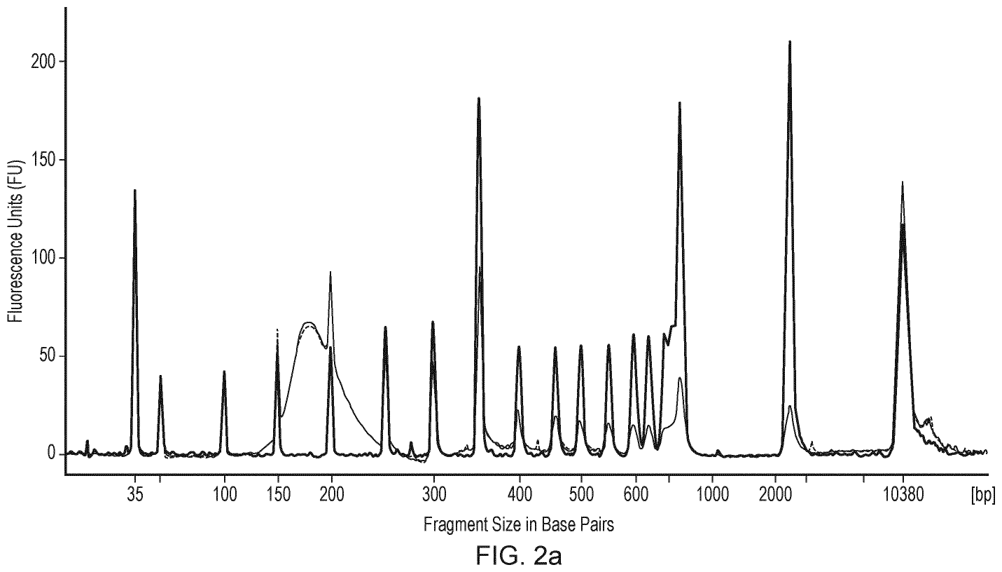

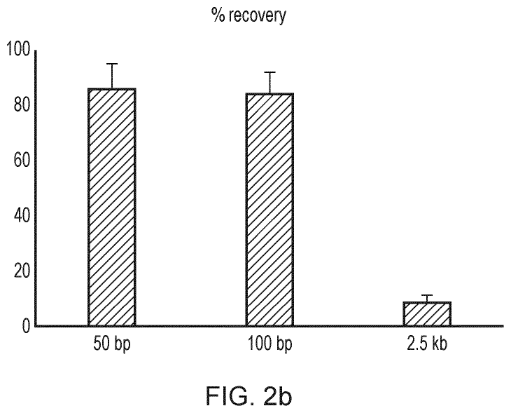

The method of the invention has been designed to maximize the recovery of small cfDNA fragments, for example as reported to be present in plasma of patients with advanced stage cancer, and to represent a fraction enriched in DNA of tumour origin. At the same time, the method design considerably reduces any co-purification of higher molecular weight gDNA that may be present, originating from lysed blood cells. This synergistic effect where small fragment recovery is elevated while large fragment recovery is depressed is demonstrated in Examples 3 and 4 described below and illustrated in FIGS. 2a and 2b respectively.

### Example 3

2 ml of plasma was obtained from the blood collected from two healthy human subjects in Streck cfDNA blood collection tubes. Both plasma samples were spiked with 50 bp DNA Ladder at a concentration of 10 ng/mL of plasma and each plasma sample was processed according to the method of the invention to extract the DNA. 1 μl of the isolated DNA from each sample was then run on High Sensitivity DNA chip in Bioanalyzer 2100. The results are as illustrated in FIG. 2a which shows a Bioanalyzer plot showing size dependent recovery of the 50 bp DNA Ladder fragments that were used to spike the plasma. As illustrated in FIG. 2a, the two independent DNA extractions are shown in blue and red trace respectively. The reference ladder equivalent to spiked input is shown in green trace. As can be seen in the plot, in both spiked-in samples, 50 bp DNA Ladder fragments corresponding to the main cfDNA peak (i.e. between ˜100 and 300 bp) and fragments ≤100 bp are efficiently recovered. Relative recovery of 50 bp DNA Ladder fragments shows minimal recovery at 2.5 kb. This example demonstrates that the cfDNA isolation method of the invention considerably reduces any co-purification of higher molecular weight gDNA contaminants originating from lysed blood cells that may be present.

### Example 4

Plasma was obtained from the blood collected from two healthy human subjects in Streck cfDNA blood collection tubes. Both plasma samples were spiked with 50 bp DNA Ladder at a concentration of 10 ng/mL of plasma and each plasma sample was processed according to the method of the invention to extract cfDNA. Percent recovery of spiked-in 50 bp DNA Ladder for selected fragments of 50 bp, 100 bp and 2.5 kbp, based on 8 independent experiments was measured. FIG. 2b shows a plot of the measurements where error bars represent standard deviation. FIG. 2b shows a recovery profile with high percent recovery for low molecular weight fragments, i.e. 50 bp and 100 bp and a low percent recovery for high molecular weight fragments of 2.5 kb.

Synergistic Effect: Elevated Recovery of Small cfDNA Fragments Along with Depressed Recovery of Larger High Molecular Weight DNA

The inventors of the current invention surprisingly found that by manipulating the relative proportion of Triton X-100, 2-propanol and GuSCN in the binding mixture, it is possible to obtain the desired DNA fragment recovery profile from plasma samples. It was found that increasing the proportion of both Triton X-100 and 2-propanol in the binding mixture, recovery of cfDNA having short fragment size improved and recovery of contaminating gDNA decreased. It was also found that increasing the amount of guanidinium ions above a certain level increases binding of higher molecular weight fragments. As described previously, the binding mixture is a combination of binding buffer, 2-propanol, aqueous suspension of magnetic beads and blood plasma.

### Effect of Varying Proportion of 2-Propanol in the Binding Mixture

### Example 5

In this experiment, the proportion of 2-propanol in the binding mixture was varied to determine the effect it had on cfDNA recovery profile. The various combinations tested are summarized in Table 6 below. Proportion of Triton X-100 was fixed at −8.8% in the binding mixture. GuSCN in the binding mixture was fixed at 2M for Prep. Nos. 10A-10D. For Prep. Nos. 10E-10H, GuSCN in the binding mixture was fixed at 2.4M.

TABLE 6

| | 2-propanol (ml) | | | | | | | |
|---|---|---|---|---|---|---|---|---|
| | 0.6 | 0.9 | 1.2 | 1.6 | 0.3 | 0.6 | 0.9 | 1.2 |
| | 2-propanol (%) | | | | | | | |
| | 9.4 | 14.2 | 18.9 | 25.2 | 4.7 | 9.4 | 14.2 | 18.9 |
| Prep. No. | 10A | 10B | 10C | 10D | 10E | 10F | 10G | 10H |
| Triton X-100 fixed | 1.50 | 1.65 | 1.80 | 2.07 | 1.39 | 1.50 | 1.65 | 1.80 |
| at ~8.8% (g) | | | | | | | | |
| Gu•SCN (g) | 4.20 | 4.55 | 4.96 | 5.70 | 4.51 | 4.87 | 5.30 | 5.81 |
| H₂O (To final 10 ml) | 5.20 | 4.75 | 4.25 | 3.50 | 5.00 | 4.65 | 4.20 | 3.75 |

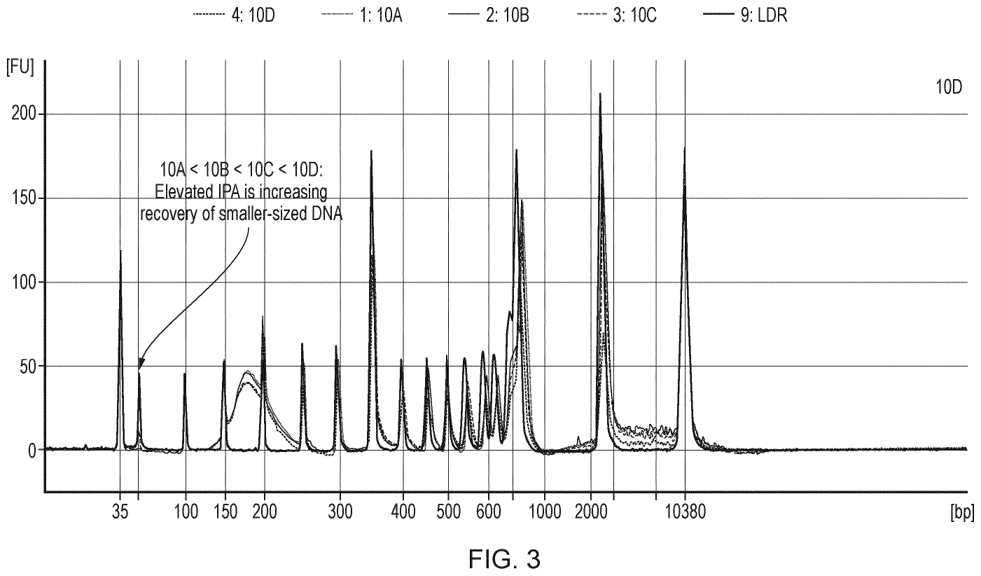

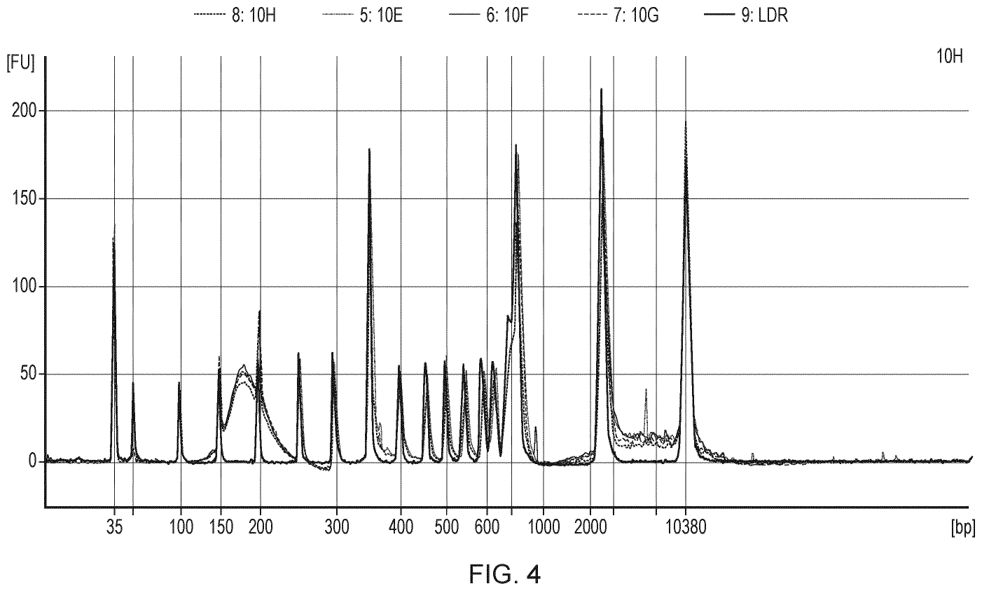

The extracted cfDNA was run on Bioanalyzer 2100 to see the recovery profile. FIG. 3 shows the Bioanalyzer plot for Prep. Nos. 10A-10D. As shown in the plot, increasing the proportion of 2-propanol in the binding mixture leads to increased recovery of smaller-sized DNA fragments. FIG. 4 shows the Bioanalyzer plot for Prep. Nos. 10E-10H with similar results. It was also observed that increased proportion of GuSCN in the binding mixture increased gDNA binding.

### Example 6

In this experiment, the effect of increasing 2-propanol from 22% to 25.2% in the binding mixture was tested while keeping GuSCN fixed at 2M and Triton X-100 fixed at 8.8% in the binding mixture.

This is summarized in Table 7 as provided below.

TABLE 7

| Prep. No. | 13A Repeat | 13G Repeat |
|---|---|---|
| 2-propanol | ~22% | ~25.2% |
| GuSCN fixed at 2M (g) | 5.1787 | 5.5623 |
| Triton X-100 fixed at | 1.924 | 2.0666 |
| 8.8% (g) | | |
| H₂O (to final 10 ml) | 4.1 | 2.6 |

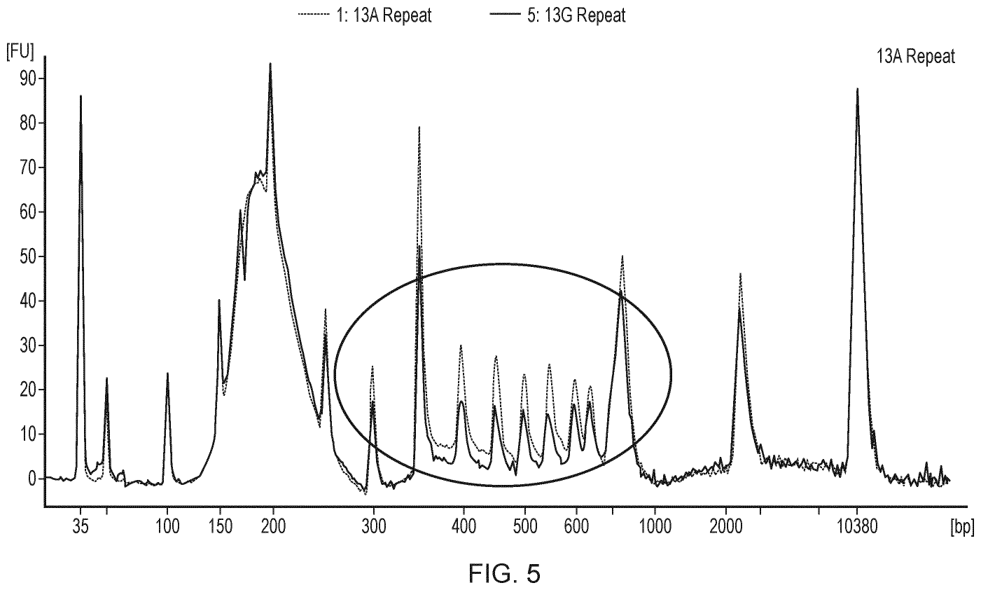

The extracted cfDNA was run on Bioanalyzer 2100 to see the recovery profile. FIG. 5 shows the Bioanalyzer plot for Prep. Nos. 13A Repeat and 13G Repeat. As shown in the plot, it was observed that increasing 2-propanol from 22% to 25.2% reduced binding of higher molecular weight DNA.

### Example 7

In this experiment, the effect of 2-propanol at 17.5%, 19%, 20.6% and 22.2% in the binding mixture was tested while keeping GuSCN fixed at 2M and Triton X-100 fixed at 11.1% in the binding mixture. This is summarized in Table 8 below.

TABLE 8

| Prep. No. | A2 | B2 | C2 | D2 |
|---|---|---|---|---|
| GuSCN | 2M | 2M | 2M | 2M |
| Triton X-100 | 11.10% | 11.10% | 11.10% | 11.10% |
| 2-Propanol | 22.20% | 20.60% | 19% | 17.50% |

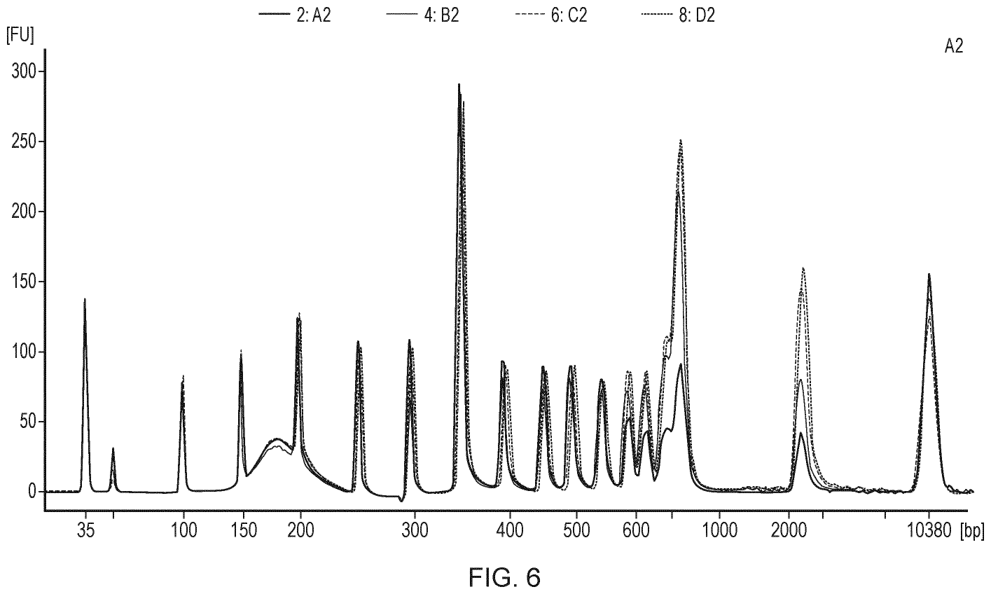

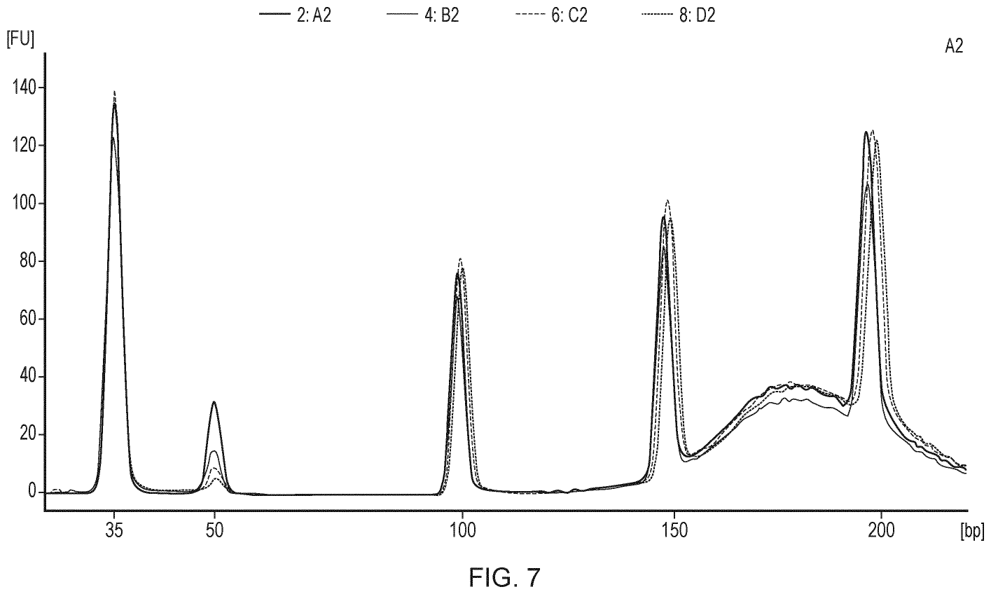

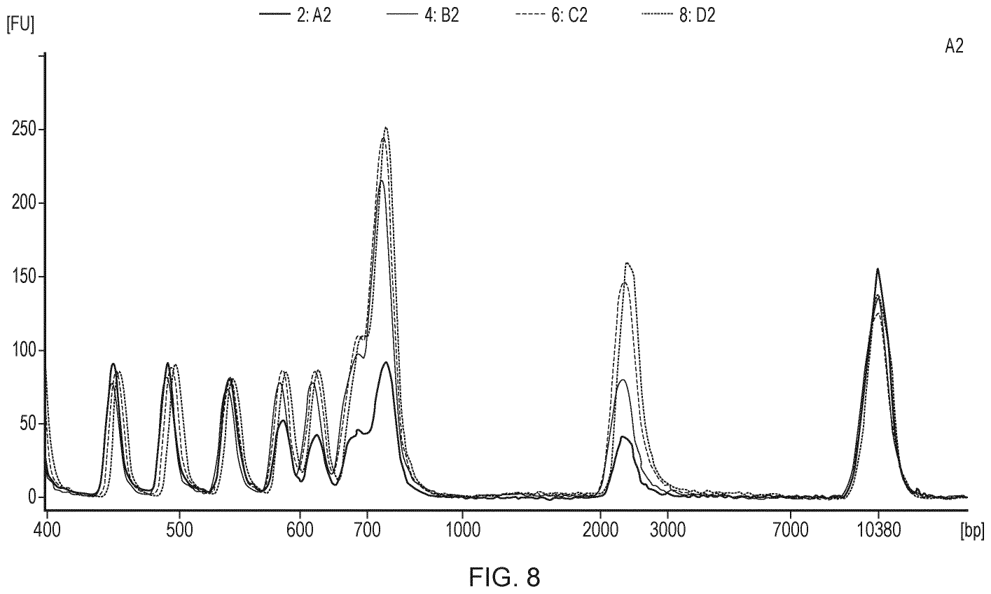

The extracted cfDNA was run on Bioanalyzer 2100 to see the recovery profile. FIG. 6 shows the Bioanalyzer plot showing effect of 2-propanol at proportions of 22.2% (A2), 20.6% (B2), 19% (C2) and 17.5% (D2) in the binding mixture on the recovery profile of extracted cfDNA. As shown in the plot, it was observed that increasing the proportion of 2-propanol from 17.5% to 22.2% in the binding mixture reduced binding of higher molecular weight DNA and increased the binding of small-sized DNA. FIG. 7 shows an enlarged view of a portion of FIG. 6 pertaining to low molecular weight DNA fragments. As shown in FIG. 7, it was observed that decreasing the proportion of 2-propanol from 22.2% to 20.6% in the binding mixture decreases 50 bp fragment recovery by at least 50%. FIG. 8 similarly shows another enlarged view of a portion of FIG. 6 pertaining to higher molecular weight DNA fragments. As shown in FIG. 8, it was observed that decreasing the proportion of 2-propanol from 22.2% to 19% in the binding mixture dramatically increases the recovery of 2.5 kb fragment.

### Effect of Varying Proportion of Triton X-100 in the Binding Mixture

### Example 8

In this experiment, the proportion of Triton X-100 in the binding mixture was varied to determine the effect it had on cfDNA recovery profile. The various combinations tested are summarized in Table 9 below. Proportions of Triton X-100 in the binding mixture were tested at 8.8% and 11.1% while keeping 2-propanol fixed at 22% and GuSCN fixed at 2M in the binding mixture.

TABLE 9

| Prep. No. | 14-II | 14-III |
|---|---|---|
| Triton X-100 | 8.8% | 11.1% |
| GuSCN fixed at 2M (g) | 5.1787 | 5.1787 |
| H₂O (to final 10 ml) | 3.3 | 2.8 |
| 2-propanol | ~22% | ~22% |

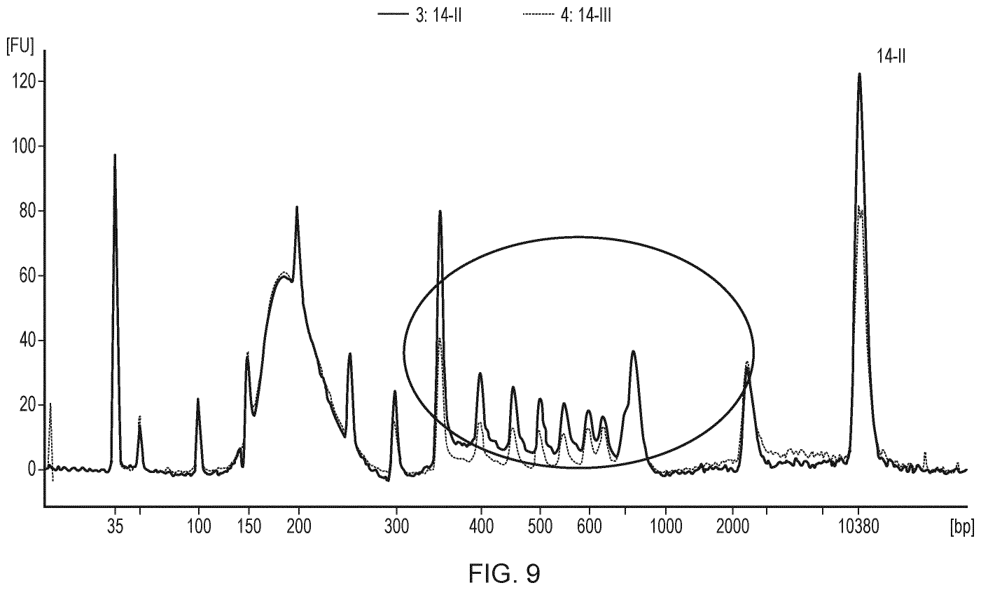

cfDNA extracts were run on Bioanalyzer 2100 to see the effect of proportion of Triton X-100 in the binding mixture on cfDNA recovery profile. FIG. 9 shows the Bioanalyzer plot where 2-propanol was fixed at ˜22%. As can be observed in FIG. 9, increasing Triton X-100 from 8.8 to 11.1% reduced binding of higher molecular weight DNA and increased small DNA fragment recovery.

### Example 9

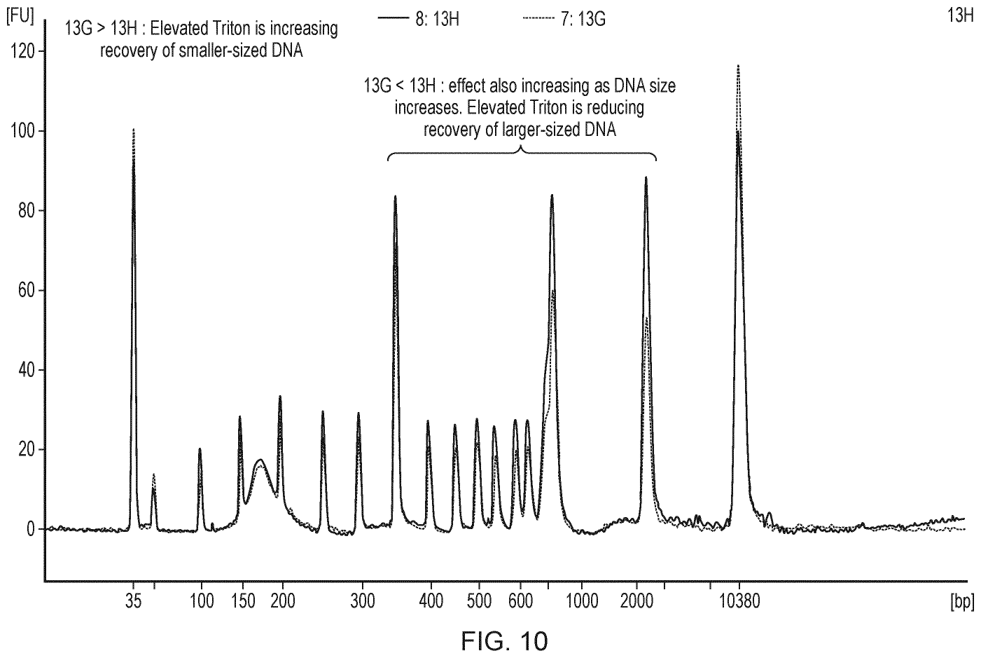

In this experiment, proportions of Triton X-100 in the binding mixture were tested at 8.8% and 4.5% while keeping 2-propanol fixed at ˜25.2% and GuSCN fixed at 2M in the binding mixture. The various combinations tested are summarized in Table 10 below. Extracts of cfDNA obtained were run on Bioanalyzer 2100 to see the effect on cfDNA recovery profile. FIG. 10 shows the Bioanalyzer plot where it was observed that elevated Triton X-100 increased recovery of smaller-sized DNA while at the same time reduced recovery of larger-sized DNA.

TABLE 10

| Prep. No. | 13G | 13H |
|---|---|---|
| Triton X-100 | 8.8% | 4.5% |
| GuSCN | 2M | 2M |
| 2-propanol | ~25.2% | ~25.2% |

### Effect of Plasma Components in the Binding Mixture

It was noted by the inventors that plasma is required in the binding mixture to achieve the desired cfDNA recovery profile. This is explained in Example 10 below.

### Example 10

In this experiment, GuSCN was fixed at 2M, Triton X-100 was fixed at 11.1% and 2-propanol was fixed at 22.2% in the binding mixture as shown in Table 11 below. Size selection of cfDNA was tested in absence of plasma. This was done by substituting plasma once with NaCl and once with PBS. Each of plasma, NaCl and PBS containing samples were spiked with 50 bp DNA Ladder to monitor size selection and DNA recovery.

TABLE 11

| Prep. No. | 2B | 3B | 4B |
|---|---|---|---|
| Input Sample | Plasma | Saline | PBS |
| Triton X-100 | 11.1% | 11.1% | 11.1% |
| GuSCN | 2M | 2M | 2M |
| 2-propanol | 22.2% | 22.2% | 22.2% |

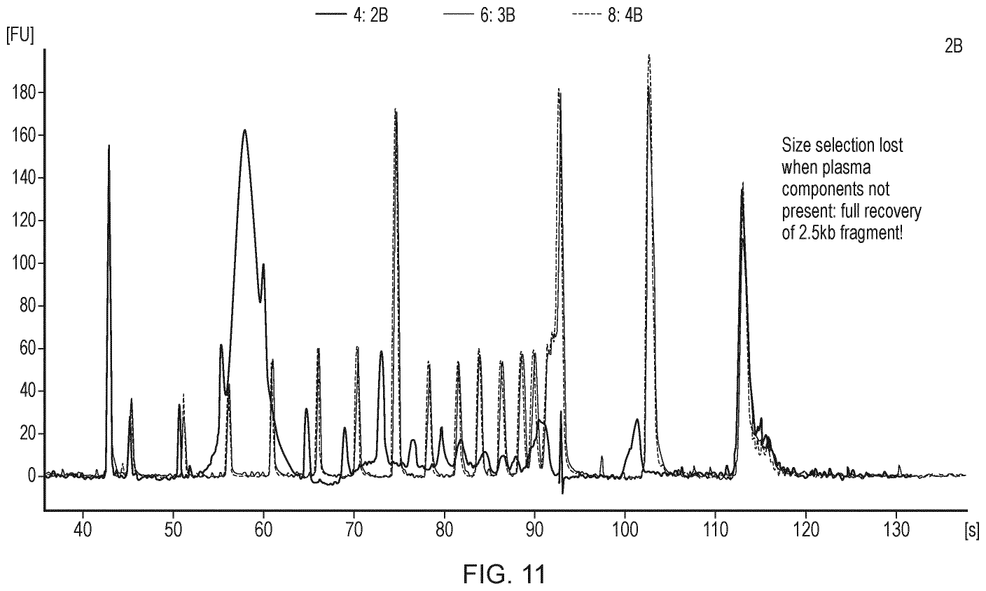

Extracted DNA was run on Bioanalyzer to see the effect of plasma on size selection. FIG. 11 shows the electropherogram where it can be seen that size selection is lost when plasma components are not present.

Scalability of the cfDNA Isolation Method

### Example 11

Plasma obtained from blood collected in Streck cfDNA blood collection tubes was spiked with 50 bp DNA Ladder at a concentration of 10 ng/ml of plasma. The spiked plasma was then processed according to the method of the invention. Four different plasma input volumes were used for this experiment (0.5 ml, 1 ml, 2 ml and 4 ml) to demonstrate the scalability of the isolation method. The elution volumes were scaled to the input plasma volume for comparable DNA concentrations in the extracts as shown below in Table 12.

TABLE 12

| | | | | |
|---|---|---|---|---|
| Prep. No. | 3D | 1A | 1C | 1E |

| | | | | | | | | |
|---|---|---|---|---|---|---|---|---|
| Plasma input | 0.5 | mL | 1 | mL | 2 | mL | 4 | mL |
| Elution volume | 7.5 | μL | 15 | μL | 30 | μL | 60 | μL |

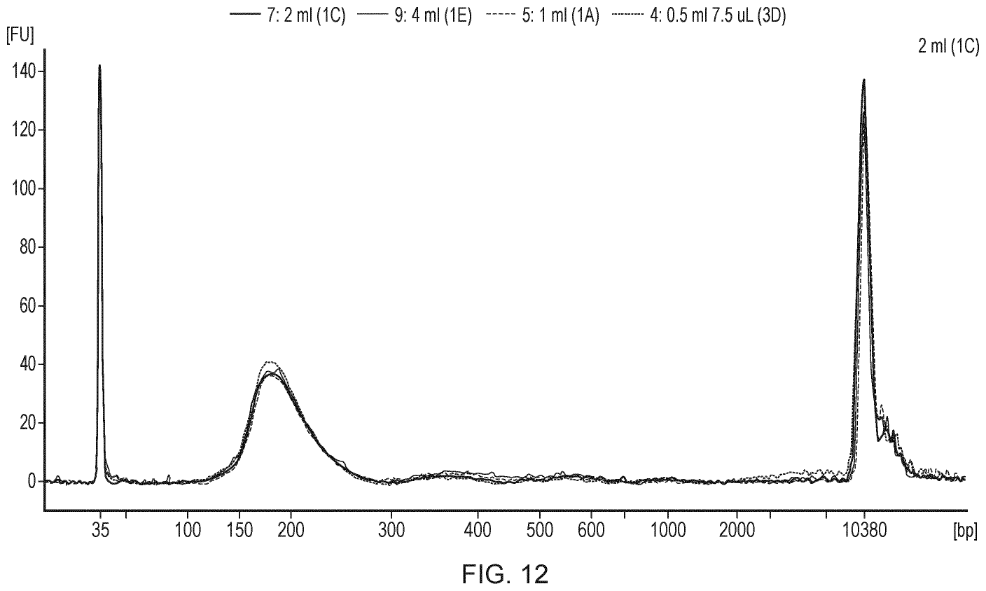

1 μl of each extract was run on High Sensitivity DNA chip on the Bioanalyzer 2100. FIG. 12 shows a Bioanalyzer 2100 plot which shows the results achieved for varying plasma input volumes (0.5 ml, 1 ml and 4 ml) compared to a standard 2 ml input. As can be inferred from the overlapping traces of the plot, the method of the invention can be used with a range of sample input volumes. Effective purification of cfDNA from plasma input volumes of 0.5 mL to 4 mL is demonstrated.

Expected Results from Plasma Collected in Standard EDTA Blood Collection Tubes

The method of the invention works best for extraction of cfDNA from blood plasma collected in Streck cfDNA blood collection tubes. However, it is possible to efficiently extract cfDNA from blood plasma collected in standard EDTA tubes as well as mentioned previously. However, in these instances, the recovery of the smaller fragments might fall below levels expected for Streck cfDNA blood collection tubes.

### Example 12

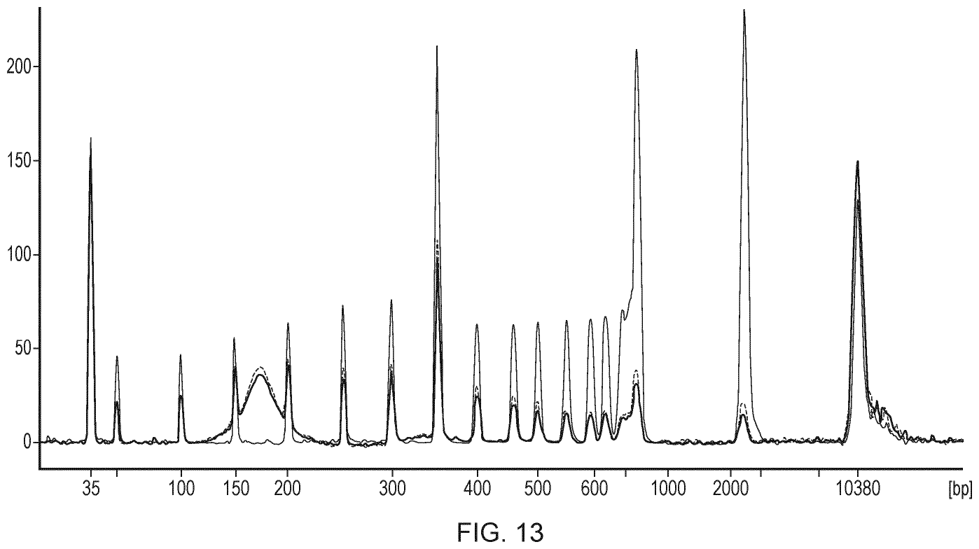

2 ml of plasma collected in standard EDTA tubes was spiked with 50 bp DNA Ladder (10 ng/mL of plasma) and processed using the cfDNA isolation method of the invention. 1 μl of the extract was run on High Sensitivity DNA chip alongside 50 bp DNA Ladder input. FIG. 13 shows a Bioanalyzer 2100 plot showing cfDNA traces and the recovery of the 50 bp DNA Ladder fragments from blood plasma collected in standard EDTA tubes (two independent extractions as depicted in blue and red trace respectively, ladder input depicted in green).

### Size Selection Advantage in Cancer Mutation Detection

The method of the invention allows for a highly efficient extraction of cfDNA and minimal carry-over of gDNA. This unique feature gives a distinctive advantage in liquid biopsy-based applications allowing for detection of mutations present at a very low level which otherwise might be missed if standard isolation methods are used. This is described in Example 13 below.

### Example 13

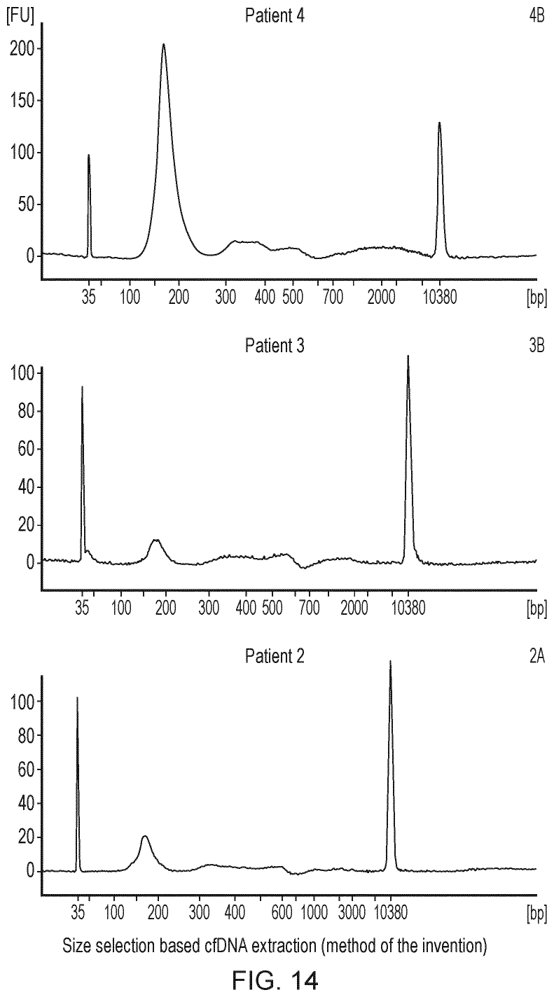

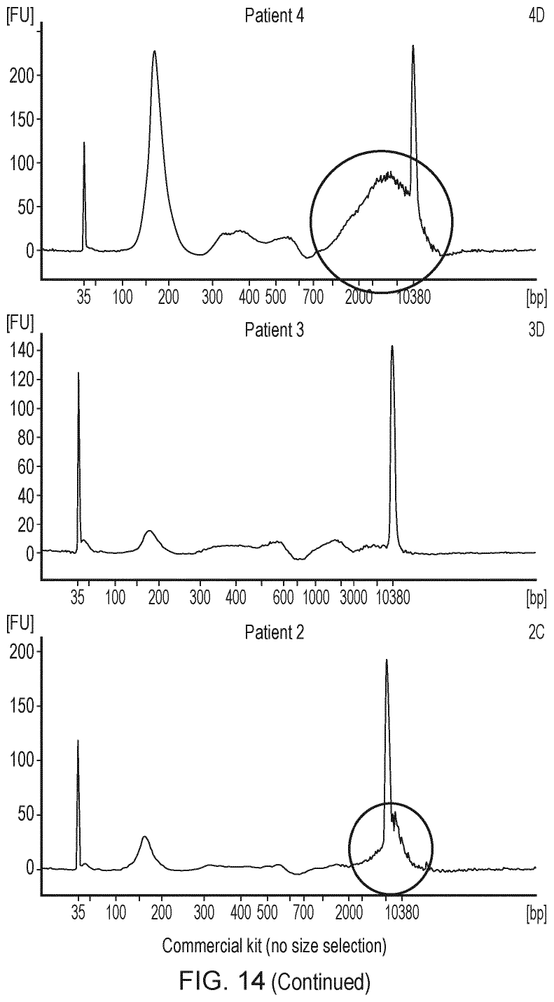

1 ml of plasma from 3 cancer patients, collected in standard EDTA blood collection tubes was obtained from commercial sources and cfDNA was isolated using the method of the invention as well as a standard commercial kit with no size selection. 1 μl of the isolated cfDNA was run on High Sensitivity DNA chip and the results were as illustrated in FIG. 14. As shown in FIG. 14, a considerable presence of high molecular weight DNA (gDNA) in patients 4 and 2 as indicated by green circles is noteworthy. The remaining eluant was concentrated and subjected to library preparation using Target Selector™ NGS Lung Panel. The results presented in Table 13 below show that for patients 4 and 2, the size selection-based extraction method gave considerably higher frequency values of detected cancer-associated mutation than a standard isolation method. Critically, in patient 3 the mutation frequency falls below assay detection level when extracted with a standard isolation method.

TABLE 13

| | Patient 4 | Patient 3 | Patient 2 |
|---|---|---|---|
| Mutation detected | BRAF V600E | TP53 R282W | BRAF V600E |
| | (gain of | (loss of | (gain of |
| | function) | function) | function) |
| Size selection-based | 0.64% | 0.61% | 2.15% |
| method (the method | | | |
| of the invention) | | | |
| Commercial kit (no | 0.27% | No variant | 0.32% |
| size selection) | | detected | |

The invention is not to be seen as limited by the embodiments and examples described above, but can be varied within the scope of the appended claims as is readily apparent to the person skilled in the art. For instance, the blood collection tubes could be standard EDTA tubes or Heparin tubes. A person skilled in the art could vary the wash buffer compositions to get essentially the same results. For example, a wash buffer could just be 70-80% aqueous ethanol. Similarly, an elution buffer could be water or any standard dilute tris-HCl or tris-EDTA buffer. It is also to be understood that the skilled person can use any suitable solid phase other than silica coated microbeads, for example, glass microbeads and glass-fibre membranes which are also DNA binding. Several alternative examples of detergents and chaotropic agents are known in the art and a skilled person could use them without departing from the scope of the claims.

1. A method for isolating cell-free DNA from liquid body sample, comprising the following steps:
a) Providing liquid body sample;
b) Adding to said sample:
a solid phase capable of binding DNA;
a binding buffer comprising a detergent and a chaotropic agent; and
2-propanol,
to form a binding mixture thereof;
c) Washing the solid phase to remove unbound material; and
d) Eluting bound cell-free DNA,
wherein the majority of the eluted DNA is <400 bp.

2. Method according to claim 1, wherein the sample is blood plasma, serum or urine.

3. Method according to claim 2, wherein blood plasma is obtained from whole blood collected in cell-free DNA stabilizing tube.

4. Method according to claim 1, wherein the detergent is a non-ionic surfactant, for example a surfactant having hydrophilic polyethylene oxide chain and an aromatic hydrocarbon lipophilic or hydrophobic group (C14 H22O(C2H4O)n where n=9-10) such as Triton X-100 or a non-ionic surfactant such as Triton X-114, Nonidet P-40 or Igepal CA-630.

5. Method according to claim 1, wherein the chaotropic agent is guanidinium thiocyanate or sodium perchlorate.

6. Method according to claim 1, wherein the sample is blood plasma, the detergent is a non-ionic surfactant such as Triton X-100 and the chaotropic agent is guanidinium thiocyanate.

7. Method according to claim 2, wherein blood plasma is at around 25-40% v/v in the said binding mixture.

8. Method according to claim 4, wherein non-ionic surfactant or Triton X-100 is at around 20-30% w/v in the said binding mixture.

9. Method according to claim 5, wherein guanidinium thiocyanate is at around 1.5-2.5 M in the said binding mixture.

10. Method according to claim 1, wherein 2-propanol is at around 15-25% v/v in the said binding mixture.

11. Method according to claim 1, wherein the solid phase comprises magnetic microbeads, preferably silica coated magnetic beads.

12. Method according to claim 11, wherein the magnetic microbeads are formulated in an aqueous suspension at 20-200 mg/ml.

13. A method for size-selective isolation of cell-free DNA from liquid body sample, comprising the following steps:
a) Providing liquid body sample;
b) Adding to said sample:
an aqueous suspension of silica coated magnetic microbeads capable of binding DNA;
a binding buffer comprising guanidinium thiocyanate and non-ionic surfactant such as Triton X-100; and
2-propanol,
to form a binding mixture thereof such that said binding mixture comprises the non-ionic surfactant such as Triton X-100 at around 20-30% w/v, guanidinium thiocyanate at around 1.5-2.5 M and 2-propanol at around 15-25% v/v;
c) Incubating the binding mixture at room temperature for about 10-30 minutes to promote binding of cell-free DNA to the magnetic microbeads;
d) Washing the magnetic microbeads with one or more wash buffers comprising ethanol;
e) Adding elution buffer to the washed magnetic beads of step d) to release the cell-free DNA bound to the magnetic microbeads in solution; and
f) Optionally analysing or quantifying the cell-free DNA obtained in step e).

14. Method according to claim 13, wherein the sample is blood plasma obtained from whole blood collected in cell-free DNA stabilizing tube.

15. Method according to claim 14, wherein blood plasma is optionally treated with proteinase K and sodium dodecyl sulfate (SDS) to form a mixture thereof and incubating the said mixture at about 55-65° C. for around 20-30 minutes.

16. Method according to claim 14, wherein blood plasma is at around 25-40% v/v in the said binding mixture.

17. Method according to claim 13, wherein the wash buffer is composed of 50% of ethanol and 50% of a solution containing guanidinium thiocyanate at around 2.0M and Triton X-100 at about 22% w/v.

18. Method according to claim 13, wherein the wash buffer is composed of 80% of ethanol and 20% of a solution containing tris-HCl at around 10 mM, ethylenediaminetetraacetic acid (EDTA) at around 1.0 mM and a polysorbate-type non-ionic surfactant such as TWEEN-20 at around 0.5% w/v.

19. Method according to claim 13, wherein the elution buffer contains tris-HCl at around 10 mM and EDTA at around the buffer being adjusted to pH 8.0.

20. Method according to claim 1, wherein said binding buffer, said microbeads and said 2-propanol are pre-mixed to give a single composite reagent before addition to said sample.

21. Method according to claim 1, wherein said microbeads and said binding buffer are added to said sample before adding said 2-propanol.

22. Method according to claim 1, wherein said binding mixture comprises:
a) Guanidinium thiocyanate preferably in the range of 1.75-2.25 M, more preferably in the range of 1.9-2.1 M, for example, approximately 2.0M;
b) Triton X-100 preferably in the range of 23-25% w/v, more preferably around 24% w/v, for example, approximately 24.1% w/v;
c) 2-propanol preferably in the range of 17-25% w/v, more preferably at around 22% w/v, for example, approximately 22.2% w/v.

23. Method according to claim 1, wherein the quantity of blood plasma used is 0.5 ml-4 ml.

24. Method according to claim 1, wherein the isolated cell-free DNA has a fragment distribution ranging from approximately 50-400 bp.

25. Use of guanidinium thiocyanate and Triton X-100 to form a binding buffer composition for size-selective binding of cell-free DNA present in blood plasma to silica-coated magnetic microbeads formulated in an aqueous suspension at 20-200 mg/ml, wherein said binding buffer is intended to be brought into contact with 2-propanol, blood plasma and magnetic microbeads to form a binding mixture comprising: around 1.5-2.5 M guanidinium thiocyanate; around 20-30% w/v of Triton X-100; around 15-25% v/v of 2-propanol; and around 25-40% v/v of blood plasma.

26. Kit comprising silica coated microbeads capable of binding 50-400 bp DNA from a liquid body sample in the presence of guanidinium thiocyanate, Triton X-100 and 2-propanol.
